(19) United States
(12) Patent Application Publication
(10) Pub. No.: US 2009/0048644 A1
(43) Pub. Date: Feb. 19, 2009

(54) System and method for providing intrabody data security on an active implantable medical device

(76) Inventors: Jeffrey E. Stahmann, Ramsey, MN (US); Kenneth P. Hoyme, Plymouth, MN (US); Keith Raymond Maile, New Brighton, MN (US); George D. Jelatis, Minneapolis, MN (US); Michael J. Timmons, Lake Elmo, MN (US); Paul Huelskamp, St. Paul, MN (US)

Correspondence Address: CASCADIA INTELLECTUAL PROPERTY, 500 UNION STREET, STE.1005, SEATTLE, WA, 98101, US

(21) Appl. No.: 11/893,251

(22) Filed: Aug. 14, 2007

(51) Int. Cl. A61N 1/08

(52) U.S. Cl. 607/60

(57) ABSTRACT

A system and method for providing intrabody data security on an active implantable medical device is presented. Data is maintained through an active implantable medical device. The data is secured on the active implantable medical device among at least one other active implantable medical device wirelessly interfaced. At least one of access to and use of the data with the other active implantable medical device is limited. Unauthorized changes to the form of the data are prevented.

### FIELD

The invention relates in general to active implantable medical devices and, specifically, to a system and method for providing intrabody data security on an active implantable medical device.

### BACKGROUND

Active implantable medical devices (AIMDs), such as defined in European Economic Community Directive 90/385/EEC, are medical devices that rely on electrical or self-provided energy. AIMDs are generally intended to be totally or partially introduced into a living body. AIMDs include both discrete devices, such as cochlear implants and insulin pumps, and multi-component devices, for instance, implantable cardiac defibrillators (ICDs) with wired intracardiac pressure sensors. Still further types of AIMDs are possible.

AIMDs provide intrabody, that is, in situ therapy or physiometric monitoring through preprogrammed autonomous or remotely controlled operation. Permanently implanted AIMDs must periodically be interfaced to external devices, such as programmers and patient-operable communicators, for diagnosis, troubleshooting, and reprogramming, and to exchange stored parametric and physiological data, while temporarily implanted AIMDs either function autonomously or operate interoperatively through an external controller.

AIMDs must balance desired capabilities against available resources, particularly power, storage, and size. Data security, for instance, serves a function secondary to the primary role of an AIMD and is only addressed implicitly. For instance, wireless data exchange provided through inductive or radio frequency telemetry relies upon the one-to-one nature of interconnections to ensure confidentiality, integrity, and authenticity.

Nevertheless, independent AIMDs can potentially interfere with one another when communicating with an external system or where components communicate intrabodily. Implicit data security alone is insufficient for ensuring that external devices are allowed to interface only to permissible AIMDs. Additionally, implicit data security fails to ensure confidentiality. Legal considerations, such as the Health Insurance Portability and Accountability Act (HIPAA) and the European Privacy Directive (EPD), mandate patient privacy be protected, especially patient health information (PHI) that identifies a specific individual with health- and medical-related information.

U.S. Pat. No. 6,223,018 issued Apr. 24, 2001 discloses an intrabody information transfer device. A transmission device includes a signal source for outputting a time varying signal that is modulated to a transmission electrode arranged in the vicinity of a human body. Another transmission electrode is connected to a reference voltage for the transmission device and is arranged outwardly from the human body. Similar separate apparatuses are provided for signal reception. Communication is provided through two absolute signal routes by means of a human-body induced electric field and via the air. However, data is exchanged in the open without data security control or protection.

U.S. Pat. No. 6,277,078 issued Aug. 21, 2001 discloses an intracardiac system that includes a pressure sensor and a sensor to collect information on blood flow into or out of a heart cavity. The sensors can be formed with or attached to a stent. The sensors are connected to a data relaying device via electrical wire. An extracorporeal processing unit collects relayed information via wired or wireless interconnect. However, data security is implicit, as data is only exchanged one-to-one between the sensors and the data relaying device, or between the data relaying device and the extracorporeal processing unit.

U.S. Pat. No. 6,764,446 issued Jul. 20, 2004 discloses an implantable pressure sensor, which measures pressure or other physiological parameters. An external transducer transmits acoustic signals into a patient's body to energize a capacitor. An acoustic transceiver converts energy between electrical and acoustic energy. The capacitor stores the electrical energy converted by the transceiver and provides electrical energy to operate the sensor, which sends pressure data as acoustic signals. Although serial numbers can be used to distinguish between multiple devices, data is only exchanged one-to-one between the sensor and the external transducer and not between interoperative implanted sensors.

U.S. Patent Application Publication No. US2002/0077673 A1, published Jun. 20, 2002, pending, and U.S. Patent Application Publication No. US2002/0177782 A1, published Nov. 28, 2002, pending, both describe an implantable sensor interfaceable via an external acoustic transducer. The sensor functions autonomously to monitor pressure or physiological parameters. The acoustic transducer transmits acoustic signals into a patient's body to interface with the implantable sensor, which downloads pressure measures recorded by the sensor. However, data is only exchanged one-to-one between the sensor and the external acoustic transducer and not between interoperative implanted sensors.

Finally, U.S. Patent Application Publication No. US2002/0082480 A1, published Jun. 27, 2002, pending, and U.S. Patent Application Publication No. US2005/0021370 A1, published Jan. 27, 2005, pending, both describe network implemented remote patient management schemes. Medical devices upload physiologic data through wireless technology, such as radio frequency telemetry, over a robust internetwork, where the data can be stored in a database, processed, analyzed, or presented for viewing. The medical devices may also communicate between one another. Medical provider access is validated or authenticated, as applicable, and data transfer over a public network is encrypted to maintain security. However, communications between medical devices remain unprotected and source trustworthiness and data integrity are assumed.

Therefore, there is a need for an approach to providing data security to AIMDs for internal intrabody and external extracorporeal data communications. Such an approach would need to secure all forms of data, including patient data, commands, and any other type of information electronically storable, exchangeable, or manipulable by or between AIMDs, or other devices.

There is a further need for an approach to ensuring the availability and the security of data to authorized devices only and to also enable multiple independent AIMDs to function without interference from either within or without a patient's body.

### SUMMARY

One embodiment provides a system and a method for providing intrabody data security on an active implantable medical device. Data is maintained through an active implantable medical device. The data is secured on the active implantable medical device among at least one other active implantable medical device wirelessly interfaced. At least one of access to and use of the data with the other active implantable medical device is limited. Unauthorized changes to the form of the data are prevented.

Still other embodiments will become readily apparent to those skilled in the art from the following detailed description, wherein are described embodiments of the invention by way of illustrating the best mode contemplated for carrying out the invention. As will be realized, the invention is capable of other and different embodiments and its several details are capable of modifications in various obvious respects, all without departing from the spirit and the scope of the present invention. Accordingly, the drawings and detailed description are to be regarded as illustrative in nature and not as restrictive.

### BRIEF DESCRIPTION OF THE DRAWINGS

FIG. 1 is a block diagram showing, by way of example, discrete and component active implantable medical devices.

FIG. 2 is a functional block diagram showing a system for providing intrabody data security on an active implantable medical device, in accordance with one embodiment.

FIG. 3 is a block diagram showing intrabody devices configured in a hierarchical communications structure for use in the system of FIG. 2.

FIG. 4 is a block diagram showing intrabody devices configured in a peer-to-peer communications structure for use in the system of FIG. 2.

FIGS. 5A-D are block diagrams showing forms of data exchange between intrabody devices for use in the system of FIG. 2.

FIG. 6 is a process flow diagram showing a method for providing intrabody data security on an active implantable medical device, in accordance with one embodiment.

FIG. 7 is a process flow diagram showing data confidentiality security control for use in the method of FIG. 6.

FIG. 8 is a process flow diagram showing data integrity security control for use in the method of FIG. 6.

FIG. 9 is a process flow diagram showing source authentication security control for use in the method of FIG. 6.

FIG. 10 is a process flow diagram showing data availability security control for use in the method of FIG. 6.

### DETAILED DESCRIPTION

Although described in this application in relation to AIMDs primarily intended for providing cardio and cardiopulmonary diagnosis, therapy, or monitoring, the embodiments described apply generally to all forms of AIMDs capable of being remotely interrogated or programmed.

### Active Implantable Medical Devices

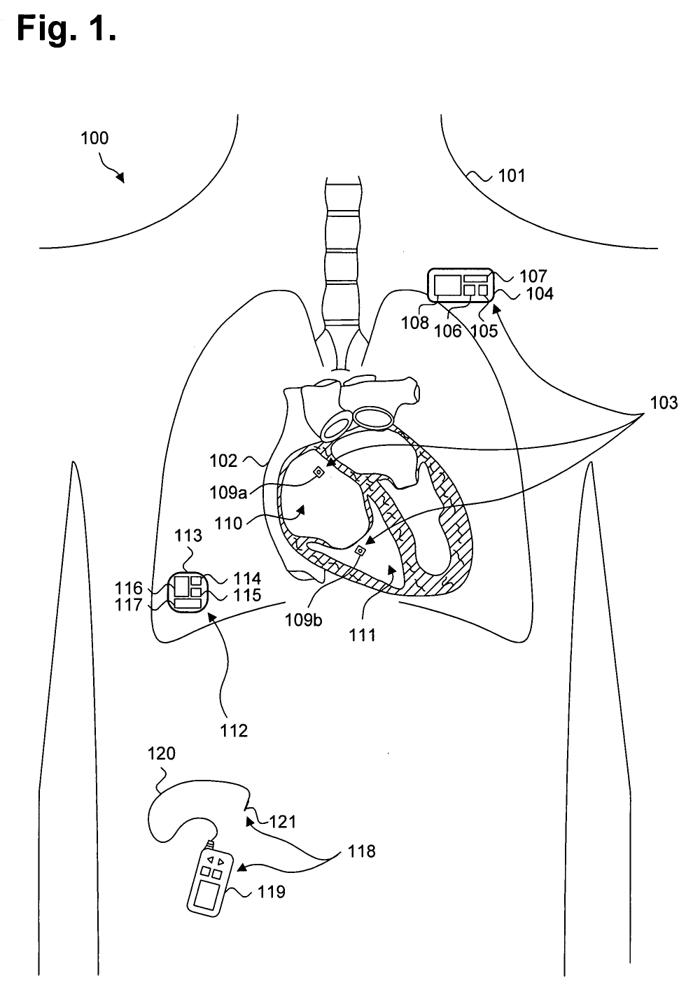

Active IMDs (AIMDs) are defined in European Economic Community Directive 90/385/EEC, the disclosure of which is incorporated by reference. In general, AIMDs rely on electrical or self-provided energy for therapy delivery, sensing, diagnosing, monitoring, and other purposes, and are intended to be totally or partially introduced, surgically or medically, into a living body or by medical intervention into a natural orifice. FIG. 1 is a block diagram showing, by way of example, discrete and component AIMDs 103, 112. Both AIMDs are implanted within the torso 100 of a human patient 101, although other implant sites, both temporary and permanent, are possible. AIMDs can also be introduced into the bodies of animals for similar purposes.

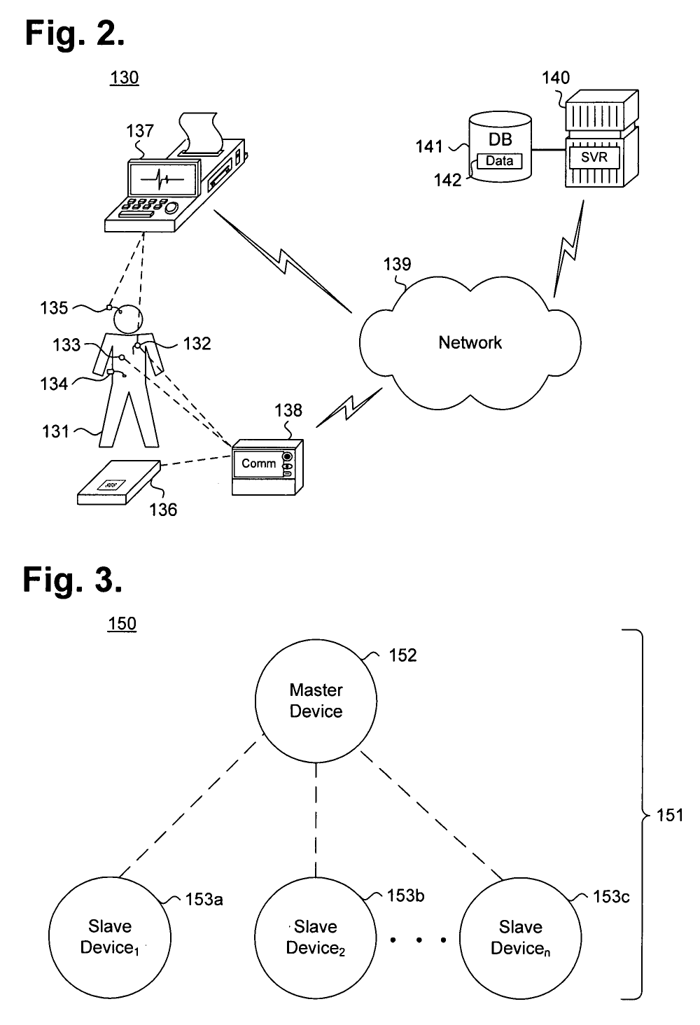

The patient 101 has received both a pair of independent AIMDs and a conventional external medical device 118. One AIMD is a multi-component AIMD 103 for intracardiac monitoring that includes separate intrabody components 104, 109a, 109b that intercommunicate wirelessly, such as through electromagnetic, acoustic, vibrational, chemical, or optical telemetry. Other forms of wireless communication are possible. Each component 104, 109a, 109b is self-powered and self-contained. A relay device 104 is implanted subdermally over the pectoralis major. The relay device 104 is wirelessly interfaced intrabodily to two remote intracardiac pressure sensors 109a, 109b that are respectively implanted in the right atrium 110 and ventricle 111 of the patient's heart 102. The monitoring device 104 can also wirelessly interface extracorporeally to an external device, such as a programmer, patient-operable communicator, or server, as further described below with reference to FIG. 2.

The housing of the relay device 104 contains control circuitry 105, telemetry circuitry 106, memory 107, and battery 108. The control circuitry 105 includes a microprocessor-based controller, signal filters, and amplifiers to process raw intracardiac data signals, which are stored into memory 107 for later retrieval and analysis by the external device. The telemetry circuitry 106 provides a wireless intrabody interface between the relay device 104 and the sensors 109a, 109b, as well as a wireless extracorporeal interface with the external device. The battery 108 provides a finite electrical power source. The sensors 109a, 109b also include a control circuitry, intrabody telemetry circuitry, battery, and memory, as well as pressure sampling circuitry (not shown). Other intrabody components and multi-component AIMDs are possible.

The other AIMD is a discrete AIMD 112 for pulmonary monitoring. A self-contained intrapulmonary sensor 113 is implanted subcutaneously over the lower lobe of the right lung. The sensor 113 can wirelessly interface extracorporeally to an external device, as further described below with reference to FIG. 2. However, communications involving the sensor 113 are distinct from those involving the relay device 104 and sensors 109a, 109b.

The housing of the sensor 113 similarly contains control circuitry 114, telemetry circuitry 115, memory 116, and battery 117. The control circuitry 114 includes a microprocessor-based controller, sampling circuits, noise filters, and amplifiers to sample and process raw intracardiac data signals, which are stored into memory 116 for later retrieval and analysis by the external device. The telemetry circuitry 115 provides a wireless interface between the sensor 113 and the external device. The battery 117 provides a finite electrical power source. Other discrete AIMDs are possible.

The conventional external medical device 118 is an insulin pump, which includes a removable stent 120 with a hollow lumen and distally oriented insulin delivery probe 121. The probe 121 is introduced subcutaneously by a caregiver or the patient for temporary therapeutic use. An insulin pump housing 119 remains outside the patient's body and communicates with the stent 120 through the hollow lumen for delivering insulin therapy. The insulin pump housing 119 includes control circuitry and battery, which execute therapy programming instructions. Communications involving the insulin pump 118 are strictly extracorporeal and therefore distinct from those involving the AIMDs 103, 112. Other external medical devices are possible.

### System

Implanted and external AIMDs perform therapy delivery, sensing, diagnosing, monitoring, and related functions, either autonomously or through remote control. Intrabody data security is necessary to ensure that these functions are performed correctly and safely, and without interference from other sources, including other AIMDs. FIG. 2 is a functional block diagram showing a system 130 for providing intrabody data security on an AIMD, in accordance with one embodiment. Intrabody data security includes protections over data when stored on an AIMD and while in transit intrabodily, particularly when transmitted wirelessly. In general, data includes patient data, commands, and any other type of information that is electronically storable, exchangeable, or manipulable by or between AIMDs, or other devices. Metadata, for instance, is data about data.

Patient data is broadly defined and includes quantitative physiometric data that has been recorded or derived from raw physiometry measured by an AIMD. Patient data also includes non-patient information, such as parametric data reporting on the status and operational characteristics of the AIMD itself, and environmental data that includes non-AIMD related information, such as the ambient temperature or time of day. Patient data further includes qualitative data values, such as subjective impressions of personal wellness or quality of life. Still further types of patient data are possible.

Commands are a specific type of instructional data, which are issued to control, effect an operational result, and communicate. Commands can be originated by an AIMD or other device, and include program or instructional codes and messages that convey directions, orders, responses, acknowledgements, and other forms of operational information to be carried out or used by an AIMD or other device. Still further types of commands are possible. Commands require security over issuance and transmittal, as well as over their integrity.

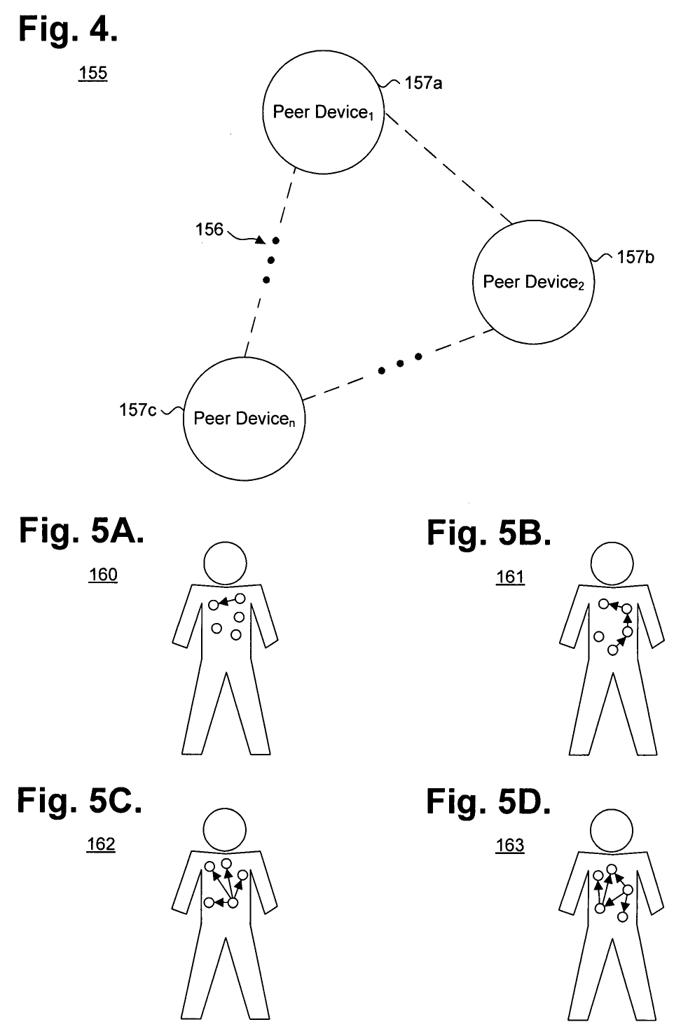

Data, whether patient data, commands, or other data, can originate with one or more multi-component or discrete AIMDs that are permanently or temporarily introduced into a patient's body. These devices include AIMDs 132, 133 that are totally introduced into a patient's body, which include therapy delivery devices, such as pacemakers, implantable cardiac defibrillators, cardiac resynchronization devices, drug pumps, and neuro-stimulators; and physiometric monitoring devices, such as cardio monitors and pulmonary monitors. These devices also include AIMDs that are partially introduced into a patient's body, which include therapy delivery devices 134, such as remotely controlled insulin pumps consisting of an extracorporeal controller and an implanted bolus delivery device, and physiometric monitoring devices 135, such as electroencephalogram recorders consisting of an extracorporeal recording device and electrodes that are placed subdurally or in the cerebral cortex. Other types of AIMDs are possible. Multi-component AIMDs can be hierarchically structured in master-slave fashion, or as peer-to-peer components, as further described below with reference to FIGS. 3 and 4, respectively. In addition, information can be exchanged between the components of a multi-component AIMD as point-to-point, end-to-end, broadcast, and multicast forms of communication, as further described below with reference to FIG. 5.

Data can also originate with conventional fully extracorporeal medical devices 136. These devices include external sensors that remain in contact with the patient's body, such as a Holter monitor, and a wide range of medical and non-medical devices that a patient can use, operate, or perform tests, such as blood pressure cuffs, weight scales, Spirometers, and the like. Other types of extracorporeal devices having patient-related purposes are possible.

Generally, those AIMDs that are either permanently introduced, or which are totally implanted require extracorporeal interfacing to interrogate or retrieve data and to provide programming over the operation of the AIMD while in situ. Extracorporeal interfacing to these types of AIMDs can be provided through conventional interrogation devices, such as programmers 137, patient-operable communicators 138, or similar devices, which are interfaced to an AIMD through wired or wireless means, such as inductive or radio frequency, or other forms of wireless telemetry based on, for example, “strong” Bluetooth or IEEE 802.11 interfacing standards. Other types of devices for AIMD interrogation and programming are possible.

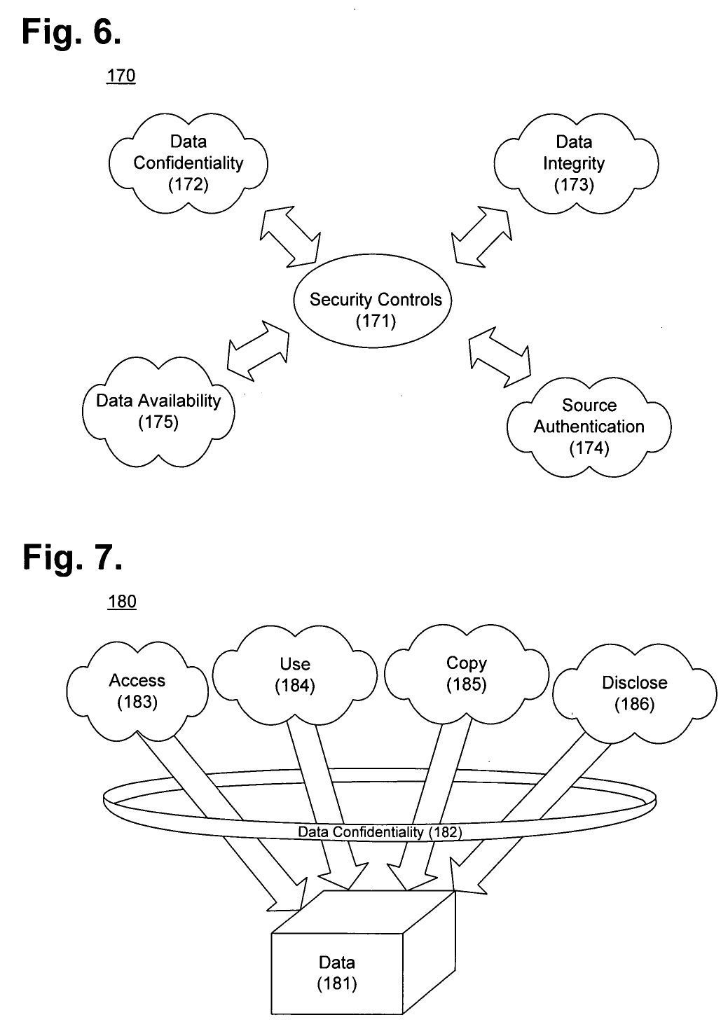

Absent data security controls, data is susceptible to a wide range of potential harms, including compromise, interception, interference, and corruption originating from within or outside of the patient's body. Data security approaches used in conventional data exchange have been applied with varying degrees of success to extracorporeal data exchange, such as between an AIMD and an external interrogation device. However, strictly intrabody communications also require data security, which can most effectively be provided through protections over data confidentiality and assurances of data integrity, source authentication, and data availability, as further described below beginning with reference to FIG. 6.

In a further embodiment, extracorporeal interfacing can be provided through a server 140, which is remotely interfaced over a network 139, either directly with an AIMD or via an intermediary interface. Structurally, the server 140 is a server-grade computing platform configured as a uni-, multi- or distributed processing system, which includes those components conventionally found in computing devices, such as, for example, a central processing unit (CPU), memory, network interface, persistent storage, and various components for interconnecting such components. The server 140 can include a database 141 or other storage means to maintain retrieved data 142 and other information for caregiver review and analysis and other authorized uses. The network 139 is based on the Transmission Control Protocol/Internet Protocol (TCP/IP) protocol suite, although other protocol suites are possible. Additionally, other network topologies and configurations are possible.

In a further embodiment, the data can be evaluated for the occurrence of one or more chronic or acute health conditions, such as described in related, commonly-owned U.S. Pat. No. 6,336,903, to Bardy, issued Jan. 8, 2002; U.S. Pat. No. 6,368,284, to Bardy, issued Apr. 9, 2002; U.S. Pat. No. 6,398,728, to Bardy, issued Jun. 4, 2002; U.S. Pat. No. 6,411,840, to Bardy, issued Jun. 25, 2002; and U.S. Pat. No. 6,440,066, to Bardy, issued Aug. 27, 2002, the disclosures of which are incorporated by reference.

In a still further embodiment, the data is extracorporeally safeguarded against unauthorized disclosure to third parties, including during collection, assembly, evaluation, transmission, and storage, to protect patient privacy and comply with recently enacted medical information privacy laws, such as the Health Insurance Portability and Accountability Act (HIPAA) and the European Privacy Directive. At a minimum, patient health information that identifies a particular individual with health- and medical-related information is treated as protectable, although other types of sensitive information in addition to or in lieu of specific patient health information could also be protectable.

### Communications Structurings

Multi-component AIMDs constitute an intrabody data network that operates independent of other devices, including extracorporeal interfacing devices, fully extracorporeal medical devices, and other AIMDs. Thus, the individual components must be structured in an organized fashion to allow secure data storage and exchange.

Hierarchical Structuring

One of the simplest forms of multi-component AIMD structurings is hierarchical. FIG. 3 is a block diagram showing intrabody devices 152, 153a-c configured in a hierarchical communications structure 150 for use in the system 130 of FIG. 2. The structure 150 is a form of tree structure with components represented by nodes and interconnections represented by paths between the nodes. Each component implements data security controls, as further described below with reference to FIG. 4, and is assigned to a node in the tree structure. One of the components is designated as a master device 152 and the other components are designated as slave devices 153a-c. The master device 152 and slave devices 153a-c form a hierarchical layer 151 and all data communications between the devices are either channeled through or arbitrated by the master device 152. Different physical components can function as the master device 152, but, absent further contention resolution mechanisms, only one component can be designated as the master device 152 at any given time. In addition, multiple hierarchical layers of successive master and slave devices could be formed, in which parent nodes function as master devices to those components represented as child nodes. Other forms of hierarchical structurings are possible.

Peer-to-Peer Structuring

Although simple, hierarchical structurings scale poorly and can unevenly tax the processing, storage, and power resources of the master device. Non-hierarchical structurings attempt to ally these shortcomings by decentralizing communication responsibility. FIG. 4 is a block diagram showing intrabody devices 157a-c configured in a peer-to-peer communications structure 155 for use in the system 130 of FIG. 2. The structure 155 is a form of fully connected, but not necessarily complete, graph with components represented by vertices and interconnections represented by edges between the vertices. Each component 157a-c implements data security controls, as further described below with reference to FIG. 4, and is assigned to a vertex in the graph. To enable non-arbitrated communications, each component 157a-c implements comparable data exchange functions and data communications are channeled along the nearest path 156 to a receiving peer device. If the structure 155 forms a complete graph, all communications must be sent directly to the receiving peer device. Otherwise, communications must be sent through successive relay by peer components. Other forms of hierarchical structurings are possible.

### Communication Forms

Data exchange can occur in one or more forms within an intrabody data network created by a multi-component AIMD. FIGS. 5A-D are block diagrams showing forms of data exchange between intrabody devices for use in the system of FIG. 2. Data security is provided to each component based on the form of communication used.

Referring first to FIG. 5A, point-to-point data exchange 160 involves a pair of components that send information directly between each other intrabodily. Source authentication control, as further described below with reference to FIG. 9, may not be required for point-to-point data exchange, as the data source is known, and presumptively trusted, based on the intrabody network topology. However, data confidentiality, data integrity, and data availability controls, as further described below respectively with reference to FIGS. 7, 8, and 10, are required.

Referring next to FIG. 5B, end-to-end data exchange 161 involves two or more components. Those components that are intermediate to the endpoints, that is, the sending and receiving components, act as data relays. Source authentication control may be required at the endpoints if point-to-point authentication is neither provided nor implicit. Data confidentiality, data integrity, and data availability controls are required.

Referring next to FIG. 5C, broadcast data exchange 162 involves a one-way transmission of information from a sending component to a plurality of receiving components. All data security controls are generally required.

Finally, referring to FIG. 5D,—multicast data exchange 163 involves a plurality of components sending information to one or more components during the same time period. All data security controls, particularly data integrity control due to the potential for data contention, are generally required. Other forms of communication are possible.

### Method

In an intrabody environment, data security is necessary to protect data when stored in an AIMD and while in transit between components or an extracorporeal device, such as a conventional interrogation device. Commands require additional security to protect their issuance and transmittal, as well as their integrity. Data security is also needed to protect against interference from fully extracorporeal medical devices and other AIMDs in operation within the same patient. FIG. 6 is a process flow diagram showing a method 170 for providing intrabody data security on an AIMD, in accordance with one embodiment. The method 170 is performed by each AIMD component, whether a discrete AIMD or as part of a multi-component AIMD, to effect intrabody data security.

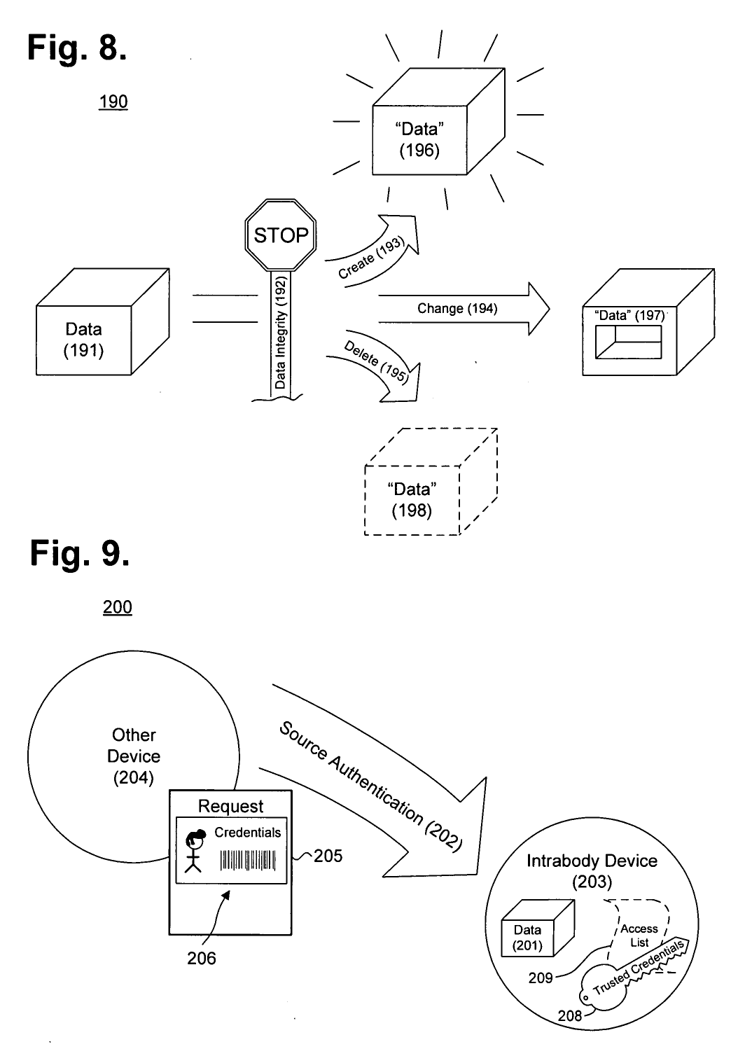

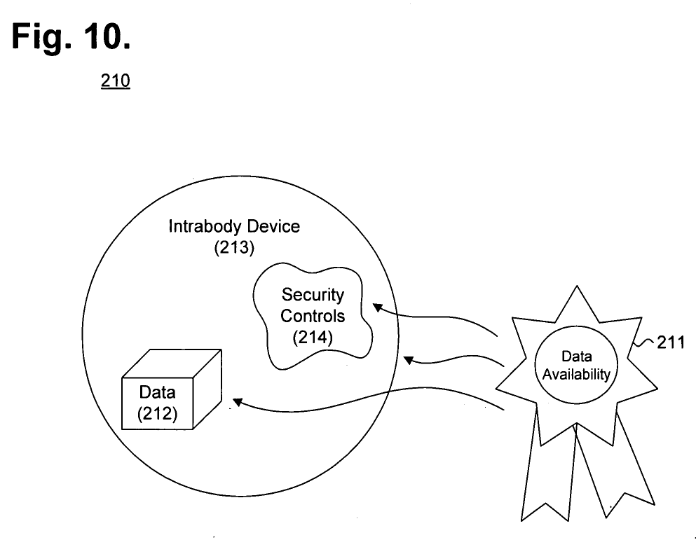

Intrabody data security is provided through a set of four security controls (operation 171). Each of the controls is necessary to ensure full data security, although the underlying functionality of the component can still be performed, albeit non-securely, if any of the controls fails or are unavailable. First, data confidentiality (operation 172) limits access to and use of data by other devices, including conventional interrogation devices, fully extracorporeal medical devices, and other AIMDs, as further described below with reference to FIG. 7. Data integrity (operation 173) prevents unauthorized changes to the form of the data, whether when stored by the component or while in transit, as further described below with reference to FIG. 8. Source authentication (operation 174) verifies the identities of other devices that attempt to interface wirelessly, as further described below with reference to FIG. 9. Finally, (operation 175) ensures that the data and the AIMD component are available and that the data security controls are functioning, as further described below with reference to FIG. 10. Other security controls either in addition to or in lieu of the foregoing security controls are possible.

Data Confidentiality

Data confidentiality ensures that data is only accessible by other devices that have been granted proper authorization. Such devices include conventional interrogation devices, fully extracorporeal medical devices, and other AIMDs, as well as other types of medical and non-medical devices. FIG. 7 is a process flow diagram showing data confidentiality security control 180 for use in the method 170 of FIG. 6. The control is implemented by both discrete AIMDs and each component of multi-component AIMDs. Additionally, comparable data security controls may be needed by an accessing device.

Data confidentiality (operation 182) is exercised over data 181, either in whole or in part, by each AIMD while stored or when in transit. Data confidentiality extends to attempts to access (operation 183), use (operation 184), copy (operation 185), or disclose (operation 186) the data 181 without proper authorization. Other operations are possible.

Data confidentiality can be introduced through several means. For instance, the AIMD can maintain an access list of authorized devices and permissible operations. As well, the data 181 can be encrypted by using symmetric encryption, such as the Advanced Encryption Standard (AES), or by using asymmetric encryption that uses public and private key pairs in a public key infrastructure, such as the Rivest, Shamir, Adleman (RSA) or Elliptic Curve Cryptography (ECC) schemes. As a result, the data 181 would only be available to those devices possessing a valid decryption cipher and key. Data confidentiality can also be achieved over a channel with limited or open access. Still further forms of data confidentiality control are possible.

Data Integrity

Whereas data confidentiality focuses on access, data integrity ensures that the form of data cannot be altered by other devices without proper authorization, whether by design or accident. For example, data could be maliciously deleted by malware, or could be unintentionally changed when in transit due to transmission error. FIG. 8 is a process flow diagram showing data integrity security control 190 for use in the method 170 of FIG. 6. The control is implemented by both discrete AIMDs and each component of multi-component AIMDs. Comparable data security controls only need be implemented by other devices when the data is being received by those devices.

Thus, data integrity (operation 192) concerns more than just the content of data 191. Data integrity involves confirming that the form of the data 191 remains as intended by the originator, which can be another device or the AIMD itself. For commands, data integrity includes protecting the issuance and transmittal of commands, as well as the integrity of the commands themselves. In basic form, data integrity typically entails including a check, marker, or other identifier with the data to enable subsequent integrity confirmation. Data integrity provides assurances that data 191 has neither been created as unauthorized new “data” 196 (operation 193), changed into unauthorized altered “data” 197 (operation 194), or deleted with resultantly lost “data” 198 (operation 195), whether by intention, inadvertence, or accident. Other operations are possible.

Data integrity can also be introduced through several means. For instance, conventional error detection and correction schemes, such as parity checking, cyclic redundancy codes, and error correction codes, can be incorporated into commands and data exchanged. Similarly, unique identifiers, such as device serial numbers, can be assigned at time of manufacture and can be included with commands and data, as well as time stamps and other protections that “lock” information against unauthorized compromise. Data integrity can also be supplied through data confidentiality mechanisms, such as cryptographic signatures included in digital certificates provided through a public key infrastructure. Still further forms of data integrity control are possible.

Source Authentication

Source authentication or, simply “authentication,” verifies claims of identity presented to an AIMD by other devices. Consequently, source authentication is needed whenever an unauthenticated device makes a request of the AIMD. FIG. 9 is a process flow diagram showing source authentication security control 200 for use in the method 170 of FIG. 6. The control is implemented by both discrete AIMDs and each component of multi-component AIMDs. Comparable data security controls must be implemented by other devices to the extent necessary to enable authentication by a recipient AIMD.

Source authentication (operation 202) is performed by an intrabody device 203 in response to a command or request for data 201 from another device 204, such as conventional interrogation devices, fully extracorporeal medical devices, and other AIMDs.

Source authentication is a form of trust mutually established between a requesting device and a receiving AIMD. Trust can be established through several means. For instance, trust can be established through data confidentiality mechanisms, such as cryptographic signatures included in a digital certificate, such as an X.509 digital certificate, which is included with a request 205 as credentials 206. The receiving intrabody device 203 would authenticate the credentials 206 against trusted credentials 208 previously received from a certificate authority or trusted agent or intermediary, such as a manufacturer. In simpler form, the intrabody device 203 could maintain an access list 209 that would identify those devices that are known and trusted. With an access list 209, the requesting device 204 would need only present a request 205 with a set of credentials 206, which could also be digitally signed. Still further forms of source authentication control are possible.

Data Availability

Data availability is less a security concern than a data access and use policy. This control ensures that data and the responsible AIMD are online with security controls that work. FIG. 10 is a process flow diagram showing data availability security control 210 for use in the method 170 of FIG. 6. The control is implemented by both discrete AIMDs and each component of multi-component AIMDs.

Data availability (operation 211) is assured if both the data 212 and associated intrabody device 213 are available to other devices, for instance, via a network 139 (shown in FIG. 2) and that the security controls 214 are functioning properly. The security controls 214 include the data confidentiality 182, data integrity 192, and source authentication 202 security controls, discussed infra, although still further security controls are possible.

Data availability can be provided as part of the security controls. Access to the data 212 and intrabody device 213 are thus refused if the security controls 214 are inoperable or otherwise offline. Still further forms of data availability control are possible.

While the invention has been particularly shown and described as referenced to the embodiments thereof, those skilled in the art will understand that the foregoing and other changes in form and detail may be made therein without departing from the spirit and scope of the invention.

What is claimed is:

1. A system for providing intrabody data security on an active implantable medical device, comprising:
data maintained through an active implantable medical device; and
security controls to secure the data on the active implantable medical device among at least one other active implantable medical device wirelessly interfaced, comprising:
a data confidentiality control to limit at least one of access to and use of the data with the other active implantable medical device; and
a data integrity control to prevent unauthorized changes to the form of the data.

2. A system according to claim 1, further comprising:
a source authentication control to verify an identity of the other active implantable medical device; and
a data availability control to ensure the data and the active implantable medical device are available and data security is functioning.

3. A system according to claim 1, further comprising:
a unique identifier assigned to the active implantable medical device prior to implantation, wherein the unique identifier is associated with each communication of the data with the active implantable medical device.

4. A system according to claim 1, wherein the use of the data further comprises one or more of copying and disclosing the data by and to the other active implantable medical device.

5. A system according to claim 1, further comprising:
an integrity check included with each communication of the data with the active implantable medical device, wherein the form of the data is confirmed by applying the integrity check to the communication.

6. A system according to claim 1, wherein the changes in the form of the data further comprise one or more of creating, modifying, and deleting the data.

7. A system according to claim 1, further comprising:
an access control to confirm the identity of the other active implantable medical device against at least one of trusted credentials and an access list of authenticated devices.

8. A system according to claim 1, further comprising:
a lock control to refuse communications with the active implantable medical device when the data is unavailable, the active implantable medical device is unavailable, or the data security controls are not functioning.

9. A system according to claim 1, further comprising:
an intrabody network structuring, comprising at least one of:
a hierarchical structuring comprising one of the active implantable medical devices configured as a master device and the other active implantable medical device configured as a slave device, wherein the master device arbitrates communications between the active implantable medical devices; and
a peer-to-peer structuring comprising the active implantable medical devices configured as peer devices, wherein communications between the active implantable medical devices are self-regulated.

10. A system according to claim 1, wherein the active implantable medical device performs a function selected from the group comprising one or more of therapy, sensing, diagnosing, and monitoring.

11. A system according to claim 1, wherein the active implantable medical device comprises one of a discrete system and an isolated component of a system that comprises a plurality of interoperating intrabody devices.

12. A system according to claim 1, wherein the data is exchanged with the other device by one or more of point-to-point, end-to-end, broadcast, and multicast communications.

13. A system according to claim 1, further comprising:
a wireless interface for the active implantable medical device to communicate with the other device through one or more of electromagnetic, acoustic, vibrational, chemical, and optical telemetry.

14. A system according to claim 1, wherein the data is selected from the group comprising commands, patient data, metadata, and other data.

15. A system according to claim 14, further comprising:
securing issuance, transmittal, and integrity of the commands.

16. A method for providing intrabody data security on an active implantable medical device, comprising:
maintaining data through an active implantable medical device; and
securing the data on the active implantable medical device among at least one other active implantable medical device wirelessly interfaced, comprising:
limiting at least one of access to and use of the data with the other active implantable medical device; and
preventing unauthorized changes to the form of the data.

17. A method according to claim 16, further comprising:
verifying an identity of the other active implantable medical device; and
ensuring the data and the active implantable medical device are available and data security is functioning.

18. A method according to claim 16, further comprising:
assigning a unique identifier to the active implantable medical device prior to implantation; and
associating the unique identifier with each communication of the data with the active implantable medical device.

19. A method according to claim 16, wherein the use of the data further comprises one or more of copying and disclosing the data by and to the other active implantable medical device.

20. A method according to claim 16, further comprising:
including an integrity check with each communication of the data with the active implantable medical device; and
confirming the form of the data by applying the integrity check to the communication.

21. A method according to claim 16, wherein the changes in the form of the data further comprise one or more of creating, modifying, and deleting the data.

22. A method according to claim 16, further comprising:
confirming the identity of the other active implantable medical device against at least one of trusted credentials and an access list of authenticated devices.

23. A method according to claim 16, further comprising:
refusing communications with the active implantable medical device when the data is unavailable, the active implantable medical device is unavailable, or the data security controls are not functioning.

24. A method according to claim 16, further comprising at least one of:
configuring one of the active implantable medical devices as a master device and the other active implantable medical device as a slave device, wherein the master device arbitrates communications between the active implantable medical devices; and
configuring the active implantable medical devices as peer devices, wherein communications between the active implantable medical devices are self-regulated.

25. A method according to claim 16, wherein the active implantable medical device performs a function selected from the group comprising one or more of therapy, sensing, diagnosing, and monitoring.

26. A method according to claim 16, wherein the active implantable medical device comprises one of a discrete system and an isolated component of a system that comprises a plurality of interoperating intrabody devices.

27. A method according to claim 16, further comprising:
exchanging the data with the other device by one or more of point-to-point, end-to-end, broadcast, and multicast communications.

28. A method according to claim 16, further comprising:
wirelessly interfacing the active implantable medical device with the other device through one or more of electromagnetic, acoustic, vibrational, chemical, and optical telemetry.

29. A method according to claim 16, wherein the data is selected from the group comprising commands, patient data, metadata, and other data.

30. A method according to claim 29, further comprising:
securing issuance, transmittal, and integrity of the commands.

31. A computer-readable storage medium holding code for performing the method according to claim 16.

32. An apparatus for providing intrabody data security on an active implantable medical device, comprising:
means for maintaining data through an active implantable medical device; and
means for securing the data on the active implantable medical device among at least one other active implantable medical device wirelessly interfaced, comprising:
means for limiting at least one of access to and use of the data with the other active implantable medical device; and
means for preventing unauthorized changes to the form of the data.
